(19) United States
(12) Patent Application Publication
(10) Pub. No.: US 2004/0147578 A1
(43) Pub. Date: Jul. 29, 2004

(54) Use of lipoaminoacids as absorption promoters in a pharmaceutical composition

(76) Inventor: Nicolas Calvet, Saint Laurent du Var (FR)

Correspondence Address: Daniel A Monaco, Drinker Biddle & Reath, One Logan Square, 18th & Cherry Streets, Philadelphia, PA, 19103-6996, US

(21) Appl. No.: 10/473,312

(22) PCT Filed: Mar. 28, 2002
(86) PCT No.: PCT/FR02/01101

(30) Foreign Application Priority Data
Mar. 28, 2001 (FR) 01/04187
May 30, 2001 (FR) 01/07051

(51) Int. Cl. A61K 31/4178; A61K 31/405; A61K 31/401; A61K 31/198

(52) U.S. Cl. 514/400; 514/419; 514/553; 514/423; 514/562; 514/563

(57) ABSTRACT

The invention concerns the use, in a pharmaceutical composition comprising at least an active principle, of at least a lipoaminoacid consisting of the combination between a fatty acid and an amino acid, the fatty acid comprising 4 to 40 carbon atoms and the amino acid may be a natural, synthetic or modified amino acid, in native or salified form; the lipoaminoacid being either an intestinal absorption promoter, or a pulmonary absorption promoter, depending on whether the composition is respectively in galenic form for oral administration or in galenic form for pulmonary administration. The invention also concerns a dispersed system comprising such a lipoaminoacid, the dispersed system being in galenic form adapted to the mode of delivery.

### TECHNICAL FIELD

The present invention relates to the field of pharmaceutical compositions and more particularly to the use of lipoaminoacids in pharmaceutical compositions as absorption promoters.

### BACKGROUND ART

A large part of the research carried out in the pharmaceutical field relates to the discovery of new active molecules, also called active principles. For a long time, the main source of new molecules was nature. Indeed, the majority of the molecules were extracted from plants.

The evolution of technologies has allowed the use of new means to obtain molecules. The first of these means is chemical synthesis. More recently, “drug design” and combinatorial chemistry have allowed the number of molecules of interest to be dramatically increased.

However, pharmaceutical research is not only limited to the discovery of new molecules. Other lines of research have been greatly developed. One of the other major lines of research is that of galenical pharmacology.

Indeed, it is now known that the pharmacological activity of an active principle does not only depend on its chemical structure and therefore on its physico-chemical properties, but also on its capacity to rejoin its active site, i.e. the point where it acts in a sufficiently large quantity. This property is called “bioavailability”.

The bioavailability of an active principle largely depends on the route used to administer it. Indeed, the active principle will be subjected to lesser or greater degradation depending on the route of administration. Therefore, it is important to choose the best route of administration for a given active principle.

The most common route of administration is the oral route. Ease of swallowing a medicine and the fact that the digestive tract is a favourable absorption site evidently account for this choice.

Other routes of administration are also used: the cutaneous route, the pulmonary route and the route relating to the mucous membranes such as the mouth, the nose, the conjunctiva (inside of the eyelids), the vagina or the rectum.

The cutaneous route is increasingly used. It is the preferred route for topical applications of medicines, notably in the form of creams, ointments, gels or patches. In some cases, this route allows the plasmatic compartment to be reached through the cutaneous barrier: this is called the transdermal passage.

Administration at the buccal, nasal, conjunctive (inside of the eyelids), vaginal and rectal mucous membranes is also advantageous. These mucous membranes have in common the fact that they consist of a membrane which lines the cavities of the body and is attached directly to the skin.

Pulmonary administration has other characteristics. The structure of the alveoli is very distinctive and allows the active principle to travel directly to the blood, which makes the method advantageous. Pulmonary administration therefore differs from the above-mentioned administrations at the mucous membranes, as well as from the cutaneous or oral administrations.

However, the bioavailability of the active principles, when the medicines are administered via these routes, is often limited.

As regards the oral route, the gastrointestinal tract is the seat for a number of enzymatic reactions. Therefore, the active principles are subjected, in the same way as food, to degradations which greatly reduce their concentration. An important stage in this tract is the crossing of the intestinal barrier, referred to as resorption. Resorption of the active principles depends on their physico-chemical characteristics.

Likewise, in the case of transdermal preparations, the active principles must cross the various layers of the epidermis. This absorption is a restricting stage which is at the origin of a greater or lesser bioavailability.

The method of administration by mucosal route, i.e. at the mucous membranes directly attached to the skin, has additional characteristics to the cutaneous application. The pHs at the mucous membranes are different. Different enzymes which are capable of degrading certain active products are also present.

As regards the pulmonary route, the presence of a large number of specific enzymes and mucosal cells involves a specific galenical pharmacology to enable the active principle to be absorbed.

It emerges therefrom that, irrespective of the route of administration, only a fraction of the quantity of administered active principle is found in the blood and rejoins its action site. Along with the problems intrinsic to the route of administration, there are other factors which influence the bioavailability of the medicine. The hepatic function is an important factor. Indeed, the liver, which acts as a filter in the body, plays a large part in the transformation of the molecules. Thus, the fraction of active principle which reaches the liver via the circulatory system, freely or attached to plasmatic molecules, is frequently larger than that which emerges therefrom. The action of the medicine is all the more limited. It has long been thought to combine the active principles with other compounds, referred to as excipients, in one formulation enabling the medicine to be formed, but also the active principle to be protected so as to limit its degradation to a maximum.

The evolution of technologies has allowed new lines of research to be set up. Thus, progress has been made from simply combining excipients and active principles to perfecting more complex systems, comprising several phases with different physico-chemical properties.

The majority of this research aims to improve the solubility of low solubility molecules. Indeed, in the case of active principles administered by oral route, resorption can only take place effectively when the molecule is dissolved in the digestive tract. The formulation plays a dominant role since it enables compounds to be chosen which, when combined with the active principle, will improve its solubility.

Several approaches have been considered to improve the solubility of low solubility compounds.

The first approach consisted of the combination of surface-active compounds in a solid or liquid state (such as sodium lauryl sulphate “SLS” or Polysorbate 80) with active principles in a solid state, in extremely varying quantities, in tablets or gelatin capsules. However, these combinations only enable the solubility of these compounds to be improved slightly and require large quantities of surfactant. Such systems can result in bad local tolerance.

A second approach consisted of the administration, directly in liquid form, of very low solubility compounds which have already been solubilised, arranged in emulsified systems most preferably of the oil-in-water type. These systems which were originally developed to ensure parenteral nutrition (Intralipid® type systems, Japanese patent No. 55.476) have been extended to the oral route for the solubilisation of hydrophobic insoluble active compounds.

Such systems relate above all to the lipophilic active molecules and have the disadvantage of being difficult to adapt to oral unit presentations.

Recently, more developed systems, forming a “preconcentrate” of surfactants and co-solvent, have been perfected. These systems form, in situ with the gastric fluids, fine emulsions which enable active principles with low water solubility, presented in unit form, to be administered by oral route. These systems such as SEDDS® (Self Emulsifying Drug Delivery System) or SMEDDS® (Self Micro-Emulsifying Drug Delivery System, European patent EP-A-0 670 715) exist equally well in a solid or liquid state. These systems are essentially intended to improve the solubility of hydrophobic active principles, by forming a fine emulsion with the gastric fluids.

One of the disadvantages of such systems is the fact that the active compounds are capable of interacting with the gastro-intestinal fluids (due to the gastric juice, pH, bile salts, blood, pancreatic lipases).

Other systems of encapsulation, semi-solid to solid, have been developed so as to improve the protection of the active principles in the gastro-intestinal environment. These are particulate micro or nano systems called liposomes. These systems are formed by stacking one or several layers of phospholipides, which contain the active principle(s) either in their centre, or in all of the structure. Such systems do not increase the solubility of the active principles but can improve the absorption of the latter.

It has been noted that after oral absorption, these systems can be sensitive to pH, pancreatic lipases and digestive juices. They then become permeable and allow the active principle(s) to be diffused from the outer layers. They appear difficult to use in unit form for adaptation to the oral route. It is more particularly developed for the injectable or topical route.

Other dispersed systems, of the oil-in-water type, form solid lipidic particles which prevent the escape of the active principle. Indeed, due to their essentially lipidic composition and their solid character, these structures are less permeable and thus restrict the diffusion processes. Given their principally lipidic composition, the quantity of solubilised molecules is relatively small. This characteristic restricts the applications to the lipophilic molecules. It can also result in a decrease in the speed of absorption of the active principles. Such compositions appear little suited to the oral unit form, given the aqueous nature of the dispersing phase.

American patent U.S. Pat. No. 5,897,876 has H/L-type emulsified systems, i.e. made up of a hydrophilic phase dispersed in a lipophilic phase. These systems allow for protection of the active principles against the destabilising action of the proteolytic enzymes and gastrointestinal fluids. However, these systems do not allow for the improvement of the absorption of the active principles. Indeed, the reduced proportion of the dispersed phase (lower than 10%) limits the quantity of active principle capable of being solubilised. Furthermore, the presence of ethanol in the hydrophilic portion can lead to a destabilisation of the system and cause irritant effects at cell level.

As a result, the composition of these systems is not sufficiently adapted to enable the absorption of the active molecules to be improved.

As regards the cutaneous route, certain systems which have been perfected are made up of a preferably oily vehicle in which the active principle is solubilised and of an adhesive part ensuring extended cutaneous contact by the oily part. Due to their structure, these systems apply only to molecules with low activity which easily cross the cutaneous barrier. Other systems use lipoamino acids or acylamino acids. Thus, in application EP 0552405A1, the acylamino acids are used as cutaneous absorption promoters in pharmaceutical compositions. These absorption promoters are not used in dispersed systems, but directly mixed into the composition. To improve the absorption of the compositions comprising these absorption promoters, the authors use an adhesive strip which is applied directly onto the skin.

In application EP 0418642A1, the acylamino acids are used as absorption promoters as part of transvaginal application. This case also does not relate to dispersed systems.

In the oral route field, which is the main route of administration, the above-mentioned systems focus their approach on the improvement of a single parameter. Indeed, certain systems only act on the stability of the active principles by restricting the destabilising effect of the gastric juices and the bile salts. Others concentrate on the improvement of the solubility and the absorption of the active principles.

Applications EP 0552405A1 and EP 0418642A1 are only based on the effect of the lipoamino acid improving the transmembranous passage of the active principle. Such compositions offer no resistance to enzymatic degradations.

No composition is employed for pulmonary use.

However, in spite of their advantages, many molecules cannot be used as active principles in these systems due to their mediocre physico-chemical properties (solubility, reactivity, etc.).

### DISCLOSURE OF THE INVENTION

The aim of the invention, therefore, is to provide a novel system using a compound capable of improving the absorption of the medicinal active principles in a pharmaceutical composition, which can be notably administered by oral, transdermic or pulmonary route. This improvement is notably observed when the compound is intimately incorporated with other constituents, thus forming a system, according to the present invention.

More precisely, the invention relates to the use, in a pharmaceutical composition comprising at least one active principle, of at least one lipoamino acid made up by the combination of a fatty acid and an amino acid, whereby the fatty acid comprises 4 to 40 carbon atoms and the amino acid can be a natural, synthetic or modified amino acid which can be in a native or salified form; the lipoamino acid being either an intestinal absorption promoter or a pulmonary absorption promoter, according to whether the composition is in a galenic form adapted to oral administration or in a galenic form adapted to lung administration respectively.

Another object of the invention is a dispersed system for pharmaceutical use including:

at least one active principle,

at least one lipoamino acid made up of the combination of a fatty acid and an amino acid, as an absorption promoter,

a dispersing phase,

a dispersed phase which is immiscible with the dispersing phase,

at least one emulsifying agent, and

a free or esterified fatty acid;

whereby the fatty acid combined with the amino acid includes 4 to 40 carbon atoms and the amino acid can be a natural, synthetic or modified amino acid, which can be in a native or salified form; the lipoamino acid being either an intestinal absorption promoter, or a pulmonary absorption promoter, according to whether the dispersed system is in a galenic form adapted to oral administration or in a galenic form adapted to lung administration respectively.

The main object of the invention is to improve the absorption of the active principles in the body following oral administration, pulmonary administration or cutaneous administration.

The unexpected feature of the invention lies in the fact that the lipoamino acids can be used in dispersed systems whilst retaining their absorption promoter property. Another unexpected feature is that these lipoamino acids, when they are made up of 4 to 40 carbon atoms, can also act as pulmonary, and especially intestinal, absorption promoters.

### DETAILED DESCRIPTION OF THE INVENTION

More precisely, the applicant observed that the lipoamino acids made up of the combination of an amino acid and a fatty acid including 4 to 40 carbon atoms, most preferably 4 to 22, were particularly advantageous absorption promoters in the case of intestinal absorption or pulmonary absorption. These absorption promoters can therefore be used as part of a pharmaceutical composition intended for oral or pulmonary administration. They can be used directly mixed with the active principle, the pharmaceutical composition being in this case in homogenous phase form. The galenical pharmacology of the composition is adapted to the method of administration.

These lipoamino acids can also be used as absorption promoters in dispersed systems for pharmaceutical use including:

at least one active principle,

at least one of these lipoamino acids,

a dispersing phase,

a dispersed phase which is immiscible with the dispersing phase,

at least one emulsifying agent, and

a fatty acid in a free or esterified state.

The advantage, in comparison to a homogenous composition, is that the dispersed systems allow the active principle to be protected against enzymatic attacks, notably at the lungs and above all at the intestines. Instead, in the dispersed systems described by the prior art, the active principle is protected from the enzymatic attacks, but the passage of the active principle at the tissue is reduced, despite the presence of an absorption promoter on occasions; thus the transmembranous passage occurs less easily than in a homogenous composition. However, the applicant observed that the lipoamino acids retain their absorption properties within a dispersed system. The dispersed systems according to the present invention therefore enable to have simultaneously the advantages of an increased resistance to enzymatic degradations and an enhanced transmembranous passage.

Since the effects of lipoamino acid absorption promoters are retained or improved within dispersed systems, dispersed systems comprising these lipoamino acids can notably be used for oral and pulmonary administrations, and also cutaneous and mucosal administrations, i.e. the buccal, nasal, conjunctive (inside of the eyelids), vaginal and rectal mucous membranes. For each of these administrations, the dispersed system must be in an appropriate galenic form for each administration.

For oral administration, the dispersed system will be in capsule or syrup form. The lipoamino acid is used in this case as an intestinal absorption promoter.

In the case of pulmonary administration, the dispersed system can notably be administered in a spray form. The lipoamino acid is used in this case as an absorption promoter at the lung.

For cutaneous applications or applications at the mucous membranes, such as the mouth, nose, the conjunctiva (inside of the eyelids), the vagina or the rectum, the dispersed system can be used in cream or gel form. The lipoamino acid is used in this case as an absorption promoter at the mucous membranes.

In homogenous compositions as in dispersed compositions, the amino acid making up the lipoamino acid is included in the natural, synthetic or modified amino acid group. Examples in the natural amino acid group include aspartic acid, glutamic acid, alanine, arginine, carnitine, choline, cysteine, glycine, histidine, isoleucine, leucine, lysine, methionine, phenylalanine, proline, ornithine, taurine, threonine, tryptophane, tyrosine, serine or valine.

The invention also relates to lipoamino acid salts. Lipoamino acid salts mean any derivative resulting from the ionic interaction between an organic or inorganic molecule with one or more reactive functions supported by the lipoamino acid.

In the case of a dispersed system, the lipoamino acid is present in a proportion between 0.01 and 50% by weight.

In the dispersed system, the lipophilic phase includes at least one lipophilic compound of natural, synthetic or modified origin. Examples include vegetable and mineral oils, waxes, fatty acids and their derivatives, fatty alcohols and their derivatives, cholesterol and its derivatives, lecithins, phospholipids, the condensation derivatives of fat bodies with sugars, polyols and ethylene and/or propylene oxide. This lipophilic compound is most preferably made up of a free or esterified fatty acid, which is present in a proportion between 0.1 and 70% by weight. According to its composition, this lipophilic phase is in a liquid, semi-solid or solid state at ambient temperature.

Likewise, the hydrophilic phase includes at least one hydrophilic compound of natural, synthetic or modified origin. Examples include water, alcohols, hydrophilic compounds such as derivatives of propylene glycol, glycerol, glycols, polyols, polymers derived from the condensation of ethylene oxide or propylene oxide, hydrocolloids. According to its composition, this hydrophilic phase is in a liquid, semi-solid or solid state at ambient temperature.

The dispersed system comprises at least an emulsifying agent of natural, synthetic or modified origin. Examples include the group of Sorbitan derivatives, alkyl polyglycosides, derivatives of ethoxylated castor oils, esters of glycerols and fatty acid, esters of glycols and fatty acids, esters of polyethylene glycol and fatty acid, esters of glycerol and fatty acid, esters of polyethylene glycol and fatty alcohol, ethylene and propylene oxide-based polymers, acrylic, vinyl polymers.

Examples 1 to 8 describe formulations of dispersed systems comprising one or more lipoamino acids (in bold) and their method of preparation. These dispersed systems are more particularly of the water-in-oil type (examples 2 to 7), i.e. with a hydrophilic dispersed phase and a lipophilic dispersing phase. By contrast, example 8 describes an oil-in-water type dispersed system, i.e. with a lipophilic dispersed phase and a hydrophilic dispersing phase.

Four of these compositions have been the object of an in vitro permeation study. These are systems comprising progesterone, aciclovir and cyclosporine A.

These studies have been carried out using the most commonly used model in in vitro permeation studies. It is the Franz cell which is made up of two compartments separated by a membrane:

a donor compartment in which the composition to be studied is placed,

a membrane preimpregnated with a lipidic solution, made up of a mixture of phospholipids,

a receiver compartment containing an aqueous solution buffered at pH 7.4 under constant agitation, in which the active principle is recovered at regular intervals. Depending on the case, the composition of the medium can vary with the physico-chemical characteristics of the molecule.

These permeation studies were carried out by comparing each dispersed system comprising the lipoamino acid(s) with a simple solution of active principle.

The results of these studies are given in comparative table form, after the formulations of the dispersed systems.

The permeation expresses an active principle quantity which diffuses through a membrane per unit of surface and time (μg/cm²/h).

The examples given below are in no way limiting.

### EXAMPLE 1

### Method of Preparation of the Dispersed Systems

The dispersed systems given in examples 2 to 7 are prepared according to the inverse emulsion principle. Example 8 is prepared according to the direct emulsion principle.

Both phases are prepared independently:

a hydrophilic phase made up of at least one hydrophilic compound and the active principle(s), heated between 40° C. and 80° C.,

a lipophilic phase made up of at least one lipophilic compound, the hydrophilic/lipophilic or surface-active emulsifier, generally a modified, natural or vegetable oil, heated between 40° C. and 80° C.

According to the fusion temperatures of the phases

a liquid/liquid dispersion

a solid/liquid dispersion

a solid dispersion

are most preferably obtained, such that these different dispersions are prepared according to traditional methods of emulsification.

### EXAMPLE 2

### Dispersed System Comprising an Anti-Acne Compound, Isotretinoin

| | |
|---|---|
| Isopropyl myristate | 31.5% |
| Oleic acid | 4.5% |
| Oleic lysine | 4% |
| Monopropylene glycol | 35% |
| Demineralised water | 3% |
| NaOH 32% | 2.5% |
| Magnesium sulphate | 1% |
| Isotretinoin | 5% |
| PEG 30 polyhydroxystearate | 13.5% |

### EXAMPLE 3

### Dispersed System Intended for the Oral Route, Comprising a Hormone, Progesterone

| | |
|---|---|
| Isopropyl myristate | 9% |
| Monopropylene glycol | 50% |
| Palmitic acid | 2.5% |
| Undecylenic glycine | 10% |
| NaOH 32% | 6.3% |
| Progesterone | 0.2% |
| Magnesium sulphate | 1% |
| Polyglyceril-2 polyhydroxystearate | 15% |
| Demineralised water | 6% |

Results of the Permeation Study:

Permeation (μg/cm²/h)

| Time (hours) | Dispersed system | Active principle |
|---|---|---|
| 0 | 0 | 0 |
| 1 | 1.5 | 0.2 |
| 2 | 2.75 | 0.4 |
| 3 | 4.25 | 0.8 |

### EXAMPLE 4

### Dispersed System Intended for the Transdermic Route, Comprising a Hormone, Progesterone

| | |
|---|---|
| Soya bean oil | 31.4% |
| Oleic acid | 10% |
| Lauroyl methionine | 5% |
| Monopropylene glycol | 30% |
| Progesterone | 0.5% |
| NaOH 32% | 3.1% |
| Magnesium sulphate | 1% |
| PEG 30 polyhydroxystearate | 15% |
| Demineralised water | 4% |

Results of the Permeation Study:

Permeation (μg/cm²/h)

| Time (hours) | Dispersed system | Active principle |
|---|---|---|
| 0 | 0 | 0 |
| 1 | 1.45 | 0.25 |
| 2 | 2.75 | 0.43 |
| 3 | 3.12 | 0.85 |

### EXAMPLE 5

### Dispersed System Intended for the Oral Route, Comprising an Anti-Viral, Aciclovir

| | |
|---|---|
| Soya bean oil | 20.3% |
| Oleic acid | 3.5% |
| Capryloyl glycine | 6.5% |
| Monopropylene glycol | 45% |
| Aciclovir | 0.5% |
| Magnesium sulphate | 0.7% |
| PEG 30 polyhydroxystearate | 14.5% |
| NaOH 32% | 4% |
| Demineralised water | 5% |

### EXAMPLE 6

### Dispersed System Intended for the Topical Route, Comprising an Anti-Viral, Aciclovir

| | |
|---|---|
| Oleic acid | 1.8% |
| Capryloyl glycine | 12% |
| Monopropylene glycol | 9.5% |
| Aciclovir | 0.5% |
| Magnesium sulphate | 0.7% |
| PEG 30 polyhydroxystearate | 4.5% |
| NaOH 32% | 21% |
| Demineralised water | 50% |

Results of the Permeation Study:

Permeation (μg/cm²/h)

| Time (hours) | Dispersed system | Active principle |
|---|---|---|
| 0 | 0 | 0 |
| 1 | 4.4 | 2.35 |
| 2 | 7.6 | 4.65 |
| 3 | 10.4 | 5.80 |

### EXAMPLE 7

### Dispersed System Intended for the Oral Route, Comprising an Immunosuppressor, Cyclosporin A

| | |
|---|---|
| Sesame oil | 6.3% |
| Oleic acid | 4.5% |
| Capryloyl glycine | 3.5% |
| Monopropylene glycol | 40% |
| Magnesium sulphate | 0.5% |
| Glucose syrup | 15% |
| Alpha tocopherol | 0.05% |
| Cyclosporin A | 2% |
| PEG 30 polyhydroxystearate | 25% |
| Demineralised water | 1% |
| 32% Sodium hydroxide solution | 2.15% |

### EXAMPLE 8

### Dispersed System of the Oil-in-Water-Type Intended for the Oral Route, Comprising an Immunosuppressor, Cyclosporin A

| | |
|---|---|
| Capryloyl glycine | 15% |
| Oleic acid | 5% |
| Monopropylene glycol | 50% |
| Cyclosporin A | 2% |
| NaOH 32% | 9% |
| Polysorbate 80 | 6.5% |
| Demineralised water | 12.5% |

Results of the Permeation Study:

Permeation (μg/cm²/h)

| Time (hours) | Dispersed system | Active principle |
|---|---|---|
| 0 | 0 | 0 |
| 1 | 3.65 | 1.5 |
| 2 | 5.4 | 2.35 |
| 3 | 7.3 | 4.25 |

Analysis of the Results of the Permeation Study:

It is noted that, irrespective of the active principle tested, the permeation is significantly greater when the active principle is contained within a dispersed system comprising one or more lipoamino acids.

These results therefore allow the conclusion to be drawn that a lipoamino acid, which is most preferably formulated in a dispersed system, allows for the improvement of the transmembranous passage of an active principle and the absorption thereof. Such a compound can therefore be used as an intestinal or pulmonary absorption promoter. It can also be used in a dispersed system while retaining its absorption promoter properties.

It is likely that the lipoamino acids become organised at the hydrophilic-lipophilic interface of the emulsions due to their amphiphilic property.

Moreover, the nature of the amino acid (free or basic acid function) involved in the lipoamino acid can contribute to the formation of ionic bonds with functions supported by the active principles, which allows for enhancement of the stability of the system, and also helps contribute towards the solubilisation of the active principles.

With the use of a dispersed system, the destabilising action of the pancreatic lipases and digestive juices, and the escape and the degradation of active principles can be avoided. Indeed, in certain cases, the external oily phase of the dispersed system prevents the migration of the gastro-intestinal juices therethrough, and the resulting degradation of the active principle and destabilisation of the dispersed system.

Furthermore, the cohesion of these dispersed systems allows for intimate contact with the dermic, pulmonary or intestinal cells, which improves the availability of the discontinuous phase, in which the active principle is organised.

1. Use in a pharmaceutical composition comprising at least one active principle, at least one lipoamino acid made up of the combination of a fatty acid and an amino acid, whereby said fatty acid comprises 4 to 40 carbon atoms and said amino acid can be a natural, synthetic or modified amino acid which can be in a native or salified form; said lipoamino acid being either an intestinal absorption promoter or a pulmonary absorption promoter, according to whether the composition is in a galenic form adapted to oral administration or in a galenic form adapted to lung administration respectively.

2. Use according to claim 1, in which said fatty acid combined with said amino acid comprises 4 to 22 carbon atoms.

3. Use according to claim 1, in which said amino acid is a natural amino acid selected from the group consisting of aspartic acid, glutamic acid, alanine, arginine, carnitine, choline, cysteine, glycine, histidine, isoleucine, leucine, lysine, methionine, phenylalanine, proline, ornithine, taurine, threonine, tryptophane, tyrosine, serine, valine.

4. Dispersed system for pharmaceutical use comprising:
at least one active principle,
at least one lipoamino acid made up of the combination of a fatty acid and an amino acid, as an absorption promoter,
a dispersing phase,
a dispersed phase which is immiscible with the dispersing phase,
at least one emulsifying agent, and
a free or esterified fatty acid;
whereby said fatty acid combined with said amino acid comprises 4 to 40 carbon atoms and said amino acid can be a natural, synthetic or modified amino acid, which can be in a native or salified form; said lipoamino acid being either an intestinal absorption promoter, or a pulmonary absorption promoter, according to whether the dispersed system is in a galenic form adapted to oral administration or in a galenic form adapted to lung administration respectively.

5. Dispersed system according to claim 4, in which said fatty acid combined with said amino acid comprises 4 to 22 carbon atoms.

6. Dispersed system according to claim 4 or 5, said amino acid being a natural amino acid selected from the group consisting of aspartic acid, glutamic acid, alanine, arginine, carnitine, choline, cysteine, glycine, histidine, isoleucine, leucine, lysine, methionine, phenylalanine, proline, ornithine, taurine, threonine, tryptophane, tyrosine, serine, valine.

7. Dispersed system according to any one of claims 4 to 6, in which said dispersing phase is a lipophilic phase and said dispersed phase is a hydrophilic phase.

8. Dispersed system according to any one of claims 4 to 7, in which said dispersing phase is a hydrophilic phase and said dispersed phase is a lipophilic phase.

9. Dispersed system according to any one of claims 4 to 8, in which the lipophilic phase comprises at least one natural, synthetic or modified lipophilic compound taken from the group made up of vegetable and mineral oils, waxes, fatty acids and their derivatives, fatty alcohols and their derivatives, cholesterol and its derivatives, lecithins, phospholipids, condensation derivatives of fat bodies with sugars, polyols and ethylene or propylene oxide, said compound being in a liquid, semi-solid or solid form at ambient temperature.

10. Dispersed system according to any one of claims 4 to 9, in which the hydrophilic phase comprises at least one natural, synthetic or modified hydrophilic compound taken from the group made up by water, alcohols, hydrophilic compounds such as derivatives of propylene glycol, glycerol, glycols, polyols, polymers derived from the condensation of ethylene oxide or propylene oxide, hydrocolloids, said hydrophilic phase being in a liquid, semi-solid or solid form at ambient temperature.

11. Dispersed system according to any one of claims 4 to 10, in which said natural, synthetic or modified emulsifying agent belongs to the group made up of Sorbitan derivatives, alkyl polyglycosides, derivatives of ethoxylated castor oils, esters of glycerol and fatty acid, esters of glycols and fatty acids, esters of polyethylene glycol and fatty acid, esters of polyglycerol and fatty acid, esters of polyethylene glycol and fatty alcohol, ethylene and propylene oxide-based polymers, acrylic, vinyl polymers.

12. Dispersed system according to any one of claims 4 to 11, in which said lipoamino acid is present in a proportion between 0.01 and 50% by weight.

13. Dispersed system according to any one of claims 4 to 12, in which said free fatty acid is present in a proportion between 0.1 and 70% by weight.

14. Dispersed system according to any one of claims 4 to 13, in which said lipoamino acid is moreover a cutaneous absorption promoter when said dispersed system is in a galenic form adapted to cutaneous application.

15. Dispersed system according to any one of claims 4 to 13, in which said lipoamino acid is moreover an absorption promoter at mucous membranes such as the mouth, the nose, the vagina, the rectum or the conjunctiva, when said dispersed system is in a galenic form adapted to an administration at these mucous membranes.
